(19) United States
(12) Patent Application Publication
(10) Pub. No.: US 2006/0089672 A1
(43) Pub. Date: Apr. 27, 2006

(54) Yarns containing filaments made from shape memory alloys

(76) Inventor: Jonathan Martinek, Hamden, CT (US)

Correspondence Address: Mark Farber, Esq.;United States Surgical, A Division of Tyco Healthcare Group, LP, 150 Glover Avenue, Norwalk, CT, 06856, US

(21) Appl. No.: 10/972,464

(22) Filed: Oct. 25, 2004

(51) Int. Cl. A61L 17/00

(52) U.S. Cl. 606/228

(57) ABSTRACT

Braids and surgical devices are made from yarns that include at least one filament made from a shape memory material.

### BACKGROUND

1. Technical Field

The present disclosure relates to yarns that contain filaments made from shape memory alloys and braided multifilaments suitably adapted for use as surgical devices made from such yarns.

2. Background of Related Art

Braided multifilaments often offer a combination of enhanced pliability, knot security and tensile strength when compared to their monofilament counterparts. The enhanced pliability of a braided multifilament is a direct consequence of the lower resistance to bending of a bundle of very fine filaments relative to one large diameter monofilament. However, a tradeoff between braid strength and pliability exists in the design of conventional braided multifilaments.

Braided multifilaments intended for the repair of body tissues should meet certain requirements: they should be substantially non-toxic, capable of being readily sterilized, they should have good tensile strength and pliability, they should also have acceptable knot-tying and knot-holding characteristics and if the braided multifilaments are of the bio-degradable variety, the degradation of the braided multifilaments should be predictable and closely controlled. Furthermore, it would be extremely beneficial if the braided multifilament could be used as a radiographic marker to assist medical personnel in monitoring the status of the implanted braid during the healing process.

### SUMMARY

The present disclosure describes yarns that contain at least one filament made from a shape memory alloy. The present disclosure also describes a heterogeneous yarn that includes a plurality of filaments made from a polymeric material and at least one filament made from a shape memory alloy. The polymeric material may be bioabsorbable or non-bioabsorbable. The heterogeneous yarns can be braided into a surgical article such as a suture or tape, or may be knitted or woven into a mesh.

The present disclosure describes a heterogeneous braid that includes a first yarn having plurality of filaments made from a polymeric material and a second yarn having at least one filament made from a shape memory alloy. The present disclosure also contemplates tapes, knits or weaves made from heterogeneous yarns including a shape memory alloy and yarns made from a polymeric material. It is also contemplated that non-woven structures such as felt can be made to include fibers of shape memory material as a reinforcement or marker.

In certain embodiments, the heterogeneous braid or the braid made from one or more heterogeneous yarns are used to form surgical devices. In other embodiments, methods for approximating two tissue surfaces are contemplated. In one embodiment, a method of closing a wound in tissue includes the steps of passing said suture through the tissue and securing the ends of said suture to approximate the tissue, wherein the suture is made from first yarns and second yarns in a braided construction wherein the first yarns include a plurality of filaments comprising a polymeric material, and the second yarns include a plurality of filaments comprising a shape memory material. In another embodiment, a method of securing soft tissue to hard tissue includes the steps of: a. providing a surgical device fabricated from first yarns and second yarns in a braided construction wherein the first yarns include a plurality of filaments comprising a polymeric material and the second yarns include a plurality of filaments comprising a shape memory material; b. passing said surgical device through the soft tissue; c. securing said surgical device to the hard tissue; and d. manipulating said surgical device (e.g., by tying a knot in the device) to approximate the soft tissue and hard tissue. In yet another embodiment, a method of approximating hard tissues is contemplated, wherein a multifilament surgical device fabricated from a heterogeneous braid made from a first yarn and a second yarn in a braided construction wherein the first yarn includes a plurality of filaments comprising a polymeric material and the second yarn includes a plurality of filaments comprising shape memory material is manipulated to approximate the hard tissues.

In yet other embodiments, the present disclosure relates to a surgical device that includes a suture anchor having at least one suture secured thereto, the suture having a braid including of a first set and a second set of continuous and discrete yarns in a braided construction. The first set of yarns are heterogeneous yarns containing first and second filaments wherein the first set of filaments are made from a polymeric material and at least one of the second filaments is made from a shape memory material

### BRIEF DESCRIPTION OF THE DRAWINGS

The above and other objects, features, and advantages will become more readily apparent from the following description, reference being made to the accompanying drawings in which:

FIG. 1 is a schematic view of a heterogeneous yarn in accordance with this disclosure;

FIGS. 2A, 2B and 2C show illustrative embodiments of braids in accordance with this disclosure;

FIG. 3 shows a needle-suture combination that includes a suture made with a heterogeneous braid in accordance with this disclosure;

FIG. 4 is a perspective view of a suture, suture anchor and associated suture anchor driver as in one embodiment described herein;

FIG. 5 is an enlarged area of detail of FIG. 4;

FIG. 6 is a perspective view of a two part suture anchor being assembled with sutures of the present disclosure;

FIG. 7 is a perspective view of the suture anchor of FIG. 6 being positioned on an anchor driver;

FIG. 8 is a perspective view, partially shown in section, of the suture driver being rotated to drive the suture anchor carrying sutures in accordance with the present disclosure into bone; and

FIG. 9 is a cross-sectional view partially shown in perspective of the suture anchor and associated sutures installed through tissue and into bone.

### DETAILED DESCRIPTION OF PREFERRED EMBODIMENTS

Filaments made from shape memory alloy are used in accordance with the present disclosure to prepare yarns that can be incorporated into a braided, knitted, woven or other structure to provide a surgical device.

A plurality of filaments is used to form a yarn. A plurality of yarns is used to form a braid, knit or weave.

A “heterogeneous yarn” is a configuration containing at least two dissimilar filaments mechanically bundled together to form a yarn. The filaments are continuous and discrete, so therefore each filament extends substantially along the entire length of the yarn and maintains its individual integrity during yarn preparation, processing and use.

Unlike a heterogeneous yarn, a “homogeneous” yarn is a configuration containing substantially similar filaments. The filaments are also continuous and discrete. Therefore each filament extends substantially along the entire length of the yarn and maintains its individual integrity during yarn preparation, processing and use.

A “heterogeneous braid” is a configuration containing at least two dissimilar yarns. The two types of yarns are intertwined in a braided construction. The yarns are continuous and discrete, so therefore each yarn extends substantially along the entire length of the braid and maintains its individual integrity during braid preparation, processing and use.

In the broadest sense, this disclosure contemplates yarns that include at least one filament made from a shape memory alloy, articles made therefrom, and their use in surgery. Suitable shape memory alloys capable of being spun into continuous filaments include, but are not limited to, nitinol (NiTi), CuZnAl, CuAlNi and FeNiAl. Methods for forming fibers from shape memory alloys are within the purview of those skilled in the art. The yarn can be a homogeneous yarn made entirely of shape memory alloy filaments. In other embodiments, the yarn is a heterogeneous yarn made from at least one shape memory alloy filament in combination with a plurality of filaments made from at least one other fiber forming material. In particularly useful embodiments, the heterogeneous yarn embodiments include a plurality of shape memory alloy filaments in combination with a plurality of filaments made from at least one polymeric material.

Some examples of polymeric materials include, but are not limited too, natural, synthetic, biodegradable, non-biodegradable and shape memory polymers. A particularly useful polymeric material may be selected from the group consisting of polyamides, polyesters, polyacrylonitrile, polyethylene, polypropylene, polyglycolic acid, polylactic acid, polydioxanone, polyepsilon-caprolactone, polytrimethylene carbonate, and combinations of such materials.

Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymers include polyhydroxy acids, such as polylactides, polyglycolides and copolymers thereof; poly(ethylene terephthalate); poly(hydroxybutyric acid); poly(hydroxyvaleric acid); poly(lactide-co-(ε-caprolactone-)); poly(glycolide-co-(ε-caprolactone)); polycarbonates, poly(pseudo amino acids); poly(amino acids); poly(hydroxyalkanoate)s; polyanhydrides; polyortho esters; and blends and copolymers thereof.

Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. A further suitable non-biodegradable fiber is ultra-high molecular weight polyethylene, available under the tradename SPECTRA® (Honeywell, Inc., Morristown, N.J.) Rapidly bioerodible polymers such as poly(lactide-co-glycolide)s, polyanhydrides, and polyorthoesters, which have carboxylic groups exposed on the external surface as the smooth surface of the polymer erodes, also can be used.

A shape memory alloy possesses the ability to remember its original shape, either after mechanical deformation, which is a one-way effect, or by cooling and heating, which is a two-way effect. This phenomenon is based on a structural phase transformation which is known as martensitic transformation.

A heterogeneous braid containing a shape memory alloy, can at anytime pre-, inter- and post-operatively be exposed to the appropriate mechanical or thermal force which transforms the shape memory alloy.

Some examples include: exposing the shape memory alloy to a force which expands the yarns and/or filaments so the interstitial spaces of the braid can be impregnated with an active agent (i.e. an antimicrobial agent, an antibiotic agent, etc.), or exposing a braided multifilament surgical device intra-operatively to a force which contracts the yarns and/or filaments and tightens the tissue closure prior to tying-off a knot, or exposing the surgical device post-operatively to a force which contracts the yarns and/or filaments and prevents a tied-knot from loosening.

Additionally, a shape memory alloy can be used as a radiographic marker. It can assist medical personnel with monitoring the status of a braided multifilament surgical device during the healing process.

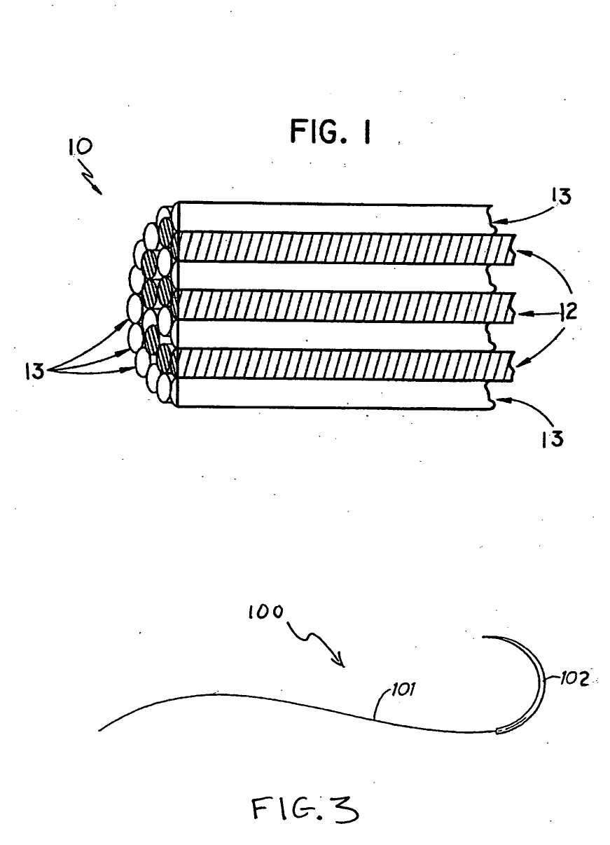

In one embodiment, a heterogeneous yarn 10 contains a plurality of two dissimilar filaments as shown in FIG. 1. First filaments 12 are made from a polymeric material and second filaments 13 are made from a shape memory alloy. A plurality of the two dissimilar filaments are commingled to form a heterogeneous yarn.

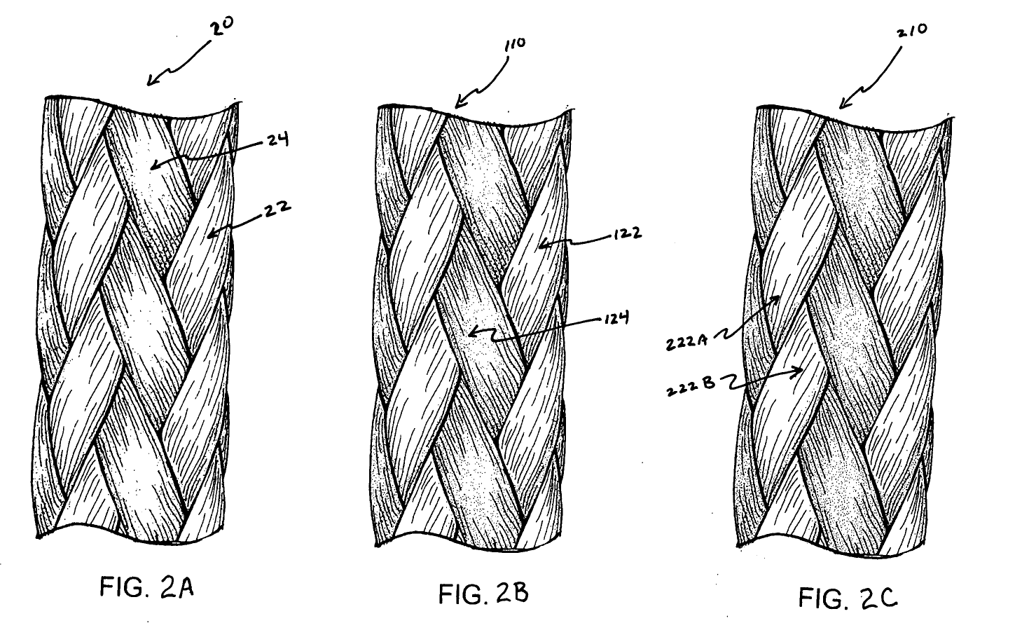

In another embodiment shown in FIG. 2A, a heterogeneous braid 20 contains two dissimilar yarns. A first yarn 22 contains a plurality of filaments made from a polymeric material. A second yarn 24 contains a plurality of filaments made from a shape memory alloy. The first and second yarns are intertwined to form a heterogeneous braid.

In still another embodiment shown in FIG. 2B, a heterogeneous braid 120 contains a heterogeneous yarn 122 and a homogeneous yarn 124. As described above, a heterogeneous yarn contains a plurality of two dissimilar filaments. Preferably, a first filament is made from a polymeric material and a second filament is made from a shape memory alloy. A homogeneous yarn contains a plurality of filaments made from any material capable of being spun into a filament. The heterogeneous yarn and the homogeneous yarn are intertwined to form a heterogeneous braid.

In yet another embodiment shown in FIG. 2C, a braid 210 contains two similar heterogeneous yarns 222A, 222B. Each heterogeneous yarn contains a plurality of two dissimilar filaments. Preferably, a first filament is made from a polymeric material and a second filament is made from a shape memory alloy. The heterogeneous yarns are intertwined to form a braid.

Particularly useful filament materials include: polymers selected from the group consisting of polyamides, polyesters, polyacrylonitrile, polyethylene, polypropylene, polyglycolic acid, polylactic acid, polydioxanone, polyepsilon-caprolactone, and polytrimethylene carbonate, and the shape memory alloy, nitinol (TiNi).

A heterogeneous braid and/or yarn can be prepared using conventional braiding technology and equipment commonly used in the textile industry, and in the medical industry for preparing multifilament sutures. Suitable braid constructions are disclosed, for example, in U.S. Pat. Nos. 3,187,752; 3,565,077; 4,014,973; 4,043,344; 4,047,533; 5,019,093; and 5,059,213, the disclosures of which are incorporated herein by reference. Illustrative flat braided structures (suitable, e.g., for tendon repair) which can be formed using the presently described heterogeneous yarns include those described in U.S. Pat. Nos. 4,792,336 and 5,318,575. Suitable mesh structures are shown and described, for example, in Hain et al. U.S. Pat. No. 5,292,328. In addition, shape memory fibers may be incorporated into non-woven structures, such as felt. One suitable non-woven structure is shown and described in Koyfinan et al. U.S. Pat. No. 5,393,534.

If desired, the surface of a filament, yarn or braid can be coated with a bioabsorbable or nonabsorbable coating to further improve the performance of the braid. For example, a braid can be immersed in a solution of a desired coating polymer in an organic solvent, and then dried to remove the solvent.

If the surface of a filament, yarn or braid is coated, then the coating composition may desirably contain bioactive materials. Some examples include: vasoactive agents, neuroactive agents, hormones, growth factors, cytokines, anaesthetics, steroids, anticoagulants, anti-inflammatories, immunomodulating agents, cytotoxic agents, prophylactic agents, antibiotics, antimicrobial, antivirals, antisense, antigens and antibodies.

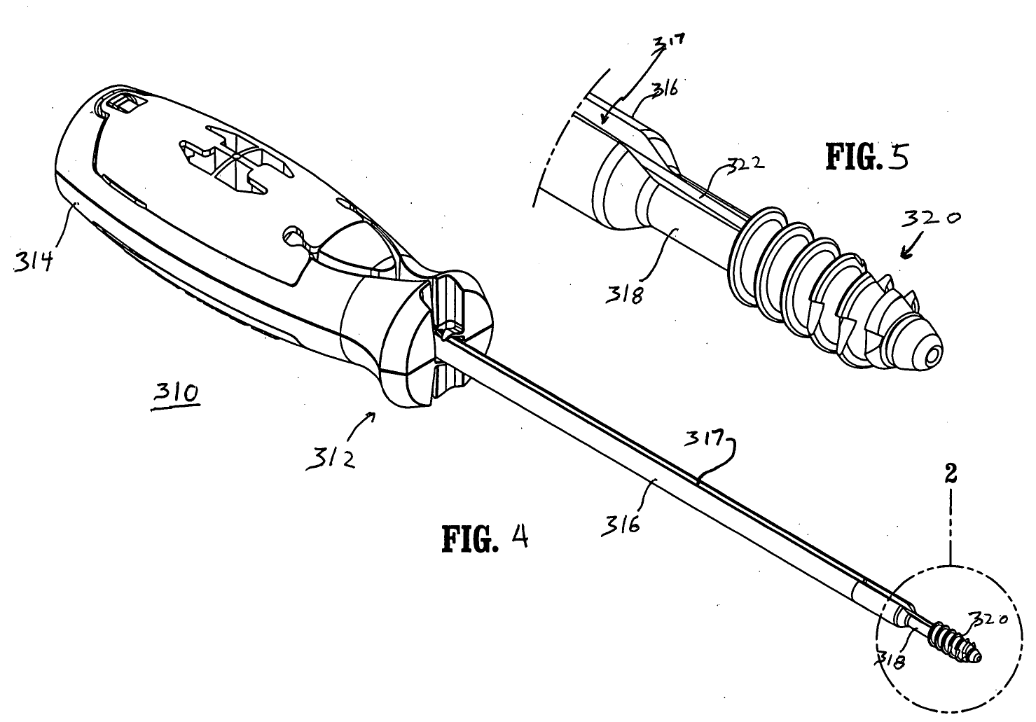

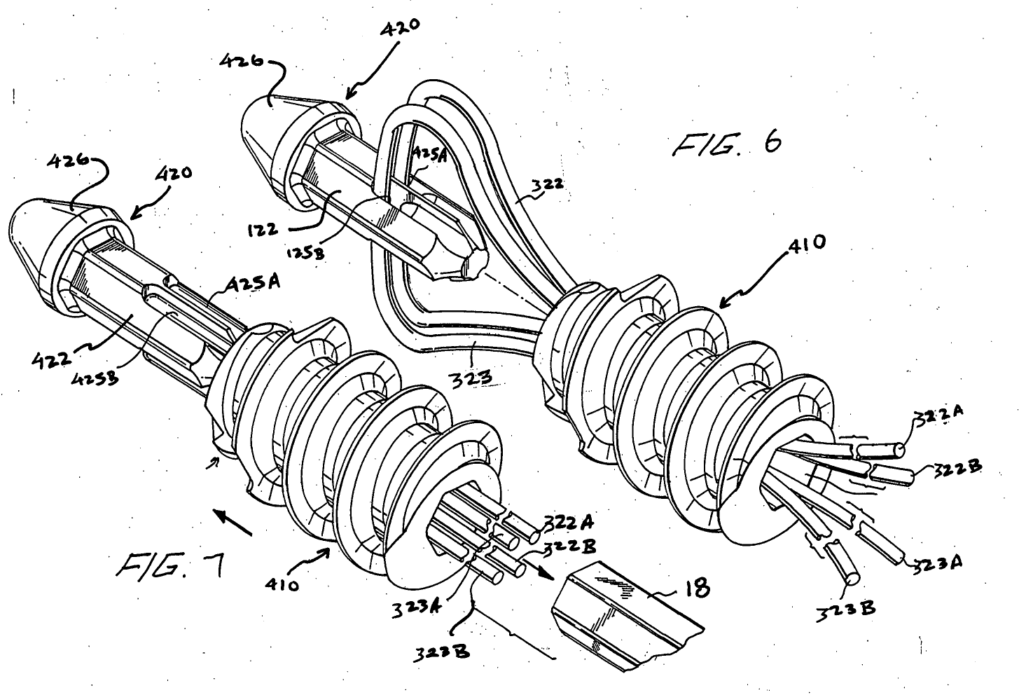

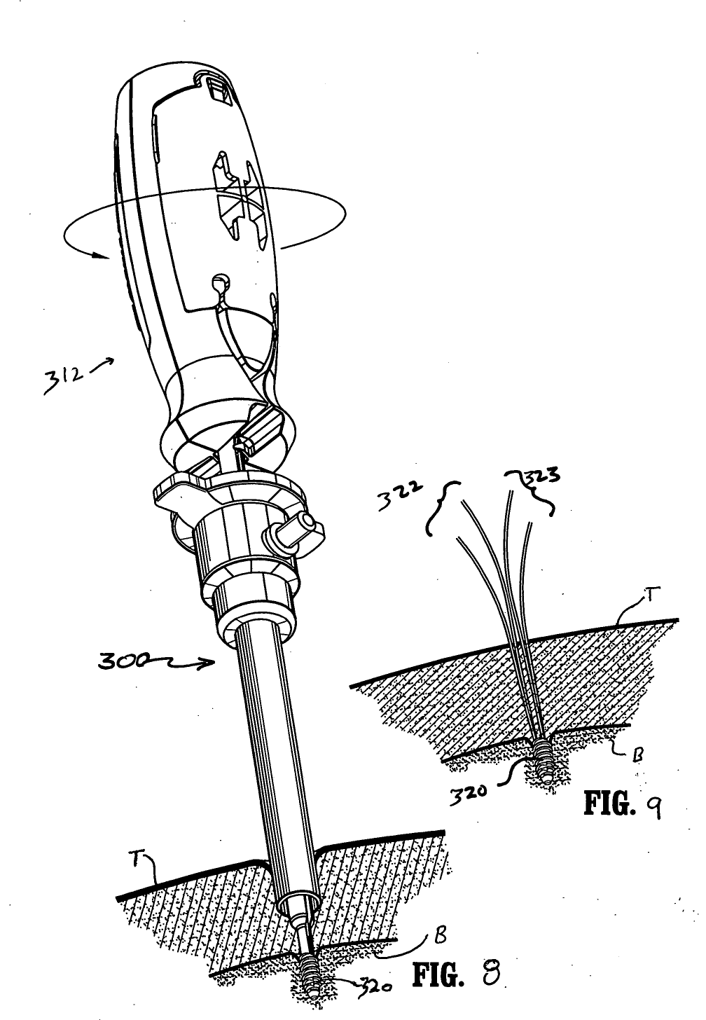

A heterogeneous braid is sterilized so it can be used for a host of medical applications, especially for use as a surgical suture, cable, tether, tape and sternal closure device, preferably attached to a needle, suture anchor, or bone anchor. For example, as shown in FIG. 3, a needle-suture combination 100 includes a suture 101 made from a heterogeneous yarn in accordance with this disclosure attached to a needle 102. A braid can be sterilized using any of the conventional techniques well known in the art.

Once sterilized, a braided multifilament surgical device, as described herein, may be used to repair wounds located between two or more soft tissues, two or more hard tissues, or at least one soft tissue and at least one hard tissue. The braided multifilament surgical device is passed through, wrapped around or secured to tissue and then the tissue is approximated by manipulating the braided multifilament surgical device, such as, for example, by tying a knot, cinching the device, applying a buckle, or the like.

In a preferred embodiment, a braid is made of heterogeneous yarns to form a surgical suture. Preferably, the heterogeneous yarns contain filaments made from a shape memory alloy and filaments made from one or more of a non-biodegradable polyester, polyethylene, polypropylene, polyamide or polyacrylanitrile. The shape memory filaments preferably comprise from about 10% to about 90% of the cross-sectional area of the heterogeneous yarns, more preferably from about 25% to 75%, and most preferably from about 25% to 50% of the heterogeneous yarns.

Most preferably, the heterogeneous yarns are made from nitinol and a non-biodegradable polyester with nitinol comprising about 10 to 90% of the braid, are preferably about 25 to 75% of the braid, and most preferably about 25 to 50% of the braid. Most preferably the heterogeneous yarns are made of nitinol and non-biodegradable polyester.

Sutures made in accordance with the foregoing description will exhibit superior strength and resistance to abrasion, and may find particular use in cardiac surgery and orthopedic surgery. With respect to orthopedic surgery in particular, the suture will be useful in securing bone under high stress and abrasion.

In a particularly useful embodiment, it is contemplated that the suture in accordance with the disclosure may be delivered in conjunction with a suture anchor delivery system and may be passed through tissue using an arthroscopic suturing instrument. Referring now to FIGS. 4 and 5, one suitable suture anchor delivery system 310 is shown having a handle 314 with an elongate shaft 316 supporting a threaded suture anchor 320 at the distal tip 318 of the shaft 316 away from the handle 314. As shown in FIG. 5, suture 322 made in accordance with the present disclosure is attached to the suture anchor 320 and is led through trough 317 in the shaft 316 (and a corresponding trough (not shown) on the other side shaft 316) to handle 314. Referring now to FIGS. 6 and 7, one method of pre-attaching a pair of sutures 322, 323 to a suture anchor is shown. As shown, suture anchor 320 consists of two parts, a hollow-threaded body portion 410 and a tip portion 420 having a shaft 422 insertable into the hollow body portion 410 and configured to receive and hold two sutures 322, 323 through transverse apertures 425A, 425B, as shown. Enlarged tip bead section 426 does not pass into or through the hollow threaded body 410, thereby retaining the suture relative to the suture anchor as the sutures 322, 323 are placed under tension by pulling on proximal ends 322A, 322B, 323A, 323B. Of course, numerous other types of suture anchors and methods of attaching sutures and in accordance with the present disclosure will occur to those skilled in the art. By way of example, the suture alternatively may be attached to a push-in-type anchor rather than a screw-in type anchor. See for example, Larsen U.S. Pat. No. 5,993,459. Preferably, the proximal ends of the suture are attached to needles (not shown) suitable for use during surgery to pass the suture through tissue and, via appropriate manipulation (e.g., knot tying and/or cinching) secure the tissue relative to the anchor.

In use during an arthroscopic procedure a cannula 300 is inserted into the joint capsule and the shaft 316 of the suture anchor delivery system is inserted through the cannula 300 to a prepared site suitable to receive suture anchor. FIG. 8 shows the shaft of the instrument inserted through cannula 430 with the suture anchor 320 inserted into bone B. The suture anchor 320 is released from the delivery system 310 leaving the sutures 322, 323 available for manipulation to secure the soft tissue T to bone B. In a further preferred embodiment the sutures are attached to needles (not shown) suitable for passing through soft tissue. The needles may be traditional suture needles suitable for use with an arthroscopic suturing instrument. One such instrument is the Arthrosew instrument (U.S.S. Sports Medicine, North Haven, Conn.) which utilizes a double ended surgical incision mender. Most preferably, the handle portion of the suture anchor delivery system includes releasable suture management members (not shown) which hold the suture needles for use. If the suture needles are to be used with a suturing device, the suture management members are configured to engage the suturing instrument in a suitable manner to transfer control of the needle to the suturing instrument. The needles and suture(s) are passed through soft tissue and the suture is manipulated, such as by forming in the suture, to secure the soft tissue relative to the suture anchor. Thereafter, the patient is closed in a suitable manner depending upon whether the procedure was conducted as an open, mini-open or closed arthroscopic approach.

In the context of a suture anchor, a suture constructed in accordance with the present disclosure provides significantly enhanced resistance to abrasion as the suture is manipulated, including drawing the suture through the suture eyelets of the suture anchor, forming knots in the suture, and cinching the knots down tightly for secure approximation of the soft tissue to bone.

Various modifications and variations of the yarns, braids and devices and uses thereof will be apparent to those skilled in the art from the foregoing detailed description. Such modifications and variations are intended to come within the scope of the following claims.

What is claimed is:

1. A surgical device comprising first yarns and multifilament second yarns in a braided construction wherein
the first yarns are made from a polymeric material; and
the second yarns comprise at least one filament made from a shape memory material.

2. The surgical device as in claim 1 wherein the polymeric material is a biodegradable polymeric material.

3. The surgical device as in claim 1 wherein the polymeric material is a non-biodegradable polymeric material.

4. The surgical device as in claim 1 wherein the shape memory material is selected from the group consisting of nitinol (TiNi), CuZnAl, CuAlNi and FeNiAl.

5. The surgical device as in claim 1 wherein the shape memory material is nitinol (TiNi).

6. The surgical device as in claim 1 wherein the first yarns include art least one filament made from at least one material selected from the group consisting of polyamides, polyesters, polyacrylonitrile, polyethylene, ultra high molecular weight polyethylene, polypropylene, polyglycolic acid, polylactic acid, polydioxanone, polyepsilon-caprolactone, and polytrimethylene carbonate.

7. The surgical device as in claim 1 wherein the first yarns are multifilament yarns.

8. The surgical device as in claim 1 wherein the braid is configured and dimensioned to form a device selected from the group consisting of a suture, mesh, sternal closure device, cable, tape and tether.

9. The surgical device as in claim 7 wherein all the filaments of the first yarn are made from a polymeric material.

10. The surgical device as in claim 1 wherein all the filaments of the second yarn are made from a shape memory material.

11. A multifilament surgical device comprising a first set and a second set of continuous and discrete yarns in a braided construction; and
each yarn from the first set contains a plurality of filaments made from at least one material selected from the group consisting of polyamides, polyesters, polyacrylonitrile, polyethylene, ultra high molecular weight polyethylene, polypropylene, polyglycolic acid, polylactic acid, polydioxanone, polyepsilon-caprolactone, and polytrimethylene carbonate; and
each yarn from the second set contains at least one filament made from shape memory material.

12. The surgical device as in claim 11 wherein the shape memory material is selected from the group consisting of nitinol (TiNi), CuZnAl, CuAlNi and FeNiAl.

13. The surgical device as in claim 11 wherein the shape memory material is nitinol (TiNi).

14. A heterogeneous yarn comprising at least one filament made from a polymeric material and at least one filament made from a shape memory material.

15. A heterogeneous yarn as in claim 14 wherein at least one filament is made from a biodegradable polymeric material.

16. A heterogeneous yarn as in claim 14 wherein at least one filament is made from a non-biodegradable polymeric material.

17. A heterogeneous yarn as in claim 14 wherein the shape memory material is selected from the group consisting of nitinol (TiNi), CuZnAl, CuAlNi and FeNiAl.

18. A surgical device comprising a heterogeneous yarn in accordance with claim 14.

19. A surgical device as in claim 18 which is selected from the group consisting of a suture, mesh, sternal closure device, cable, tape and tether.

20. A sterile braid comprising a heterogeneous yarn in accordance with claim 14.

21. A multifilament surgical device comprising a plurality of heterogeneous yarn, each heterogeneous yarn including first and second filaments, the plurality of yarns being in a braided construction wherein;
the first filaments are made from at least one fiber-forming material selected the group consisting of polyamides, polyesters, polyacrylonitrile, polyethylene, polypropylene, polyglycolic acid, polylactic acid, polydioxanone, polyepsilon-caprolactone, and polytrimethylene carbonate; and
the second filaments are made from a shape memory material.

22. A multifilament surgical device as in claim 21 wherein the shape memory material is selected from the group consisting of nitinol (TiNi), CuZnAl, CuAlNi and FeNiAl.

23. A multifilament surgical device as in claim 21 wherein the shape memory material is nitinol (NiTi).

24. A multifilament surgical device comprising a first set and a second set of continuous and discrete heterogeneous yarns, each of the heterogeneous yarns containing first and second continuous and discrete filaments,
the first filaments being made from a polymeric material; and
the second filaments being made from a shape memory material.

25. A multifilament surgical device of claim 24 wherein the first filaments are made from a biodegradable polymeric material

26. A multifilament surgical device of claim 24 wherein the first filaments are made from a non-biodegradable polymeric material.

27. A multifilament surgical device of claim 24 wherein the shape memory material is selected from the group consisting of nitinol (TiNi), CuZnAl, CuAlNi and FeNiAl.

28. A multifilament surgical device of claim 24, wherein the shape memory material is nitinol (NiTi).

29. A multifilament surgical device of claim 24 wherein the heterogeneous yarns are formed into a device selected from the group consisting of a suture, mesh, a sternal closure device, a cable, a tape and a tether.

30. A multifilament surgical device comprising a braid including a first set and a second set of continuous and discrete yarns in a braided construction;
the first set of yarns being heterogeneous yarns containing first and second filaments wherein:
the first set of filaments are made from a polymeric material; and
at least one of the second filaments is made from a shape memory material.

31. A multifilament surgical device as in claim 30 wherein the second set of yarns are homogeneous yarns.

32. A multifilament surgical device as in claim 30 wherein the shape memory material is selected from the group consisting of nitinol (TiNi), CuZnAl, CuAlNi and FeNiAl.

33. A multifilament surgical device as in claim 30 wherein the shape memory material is nitinol (NiTi).

34. A multifilament surgical device as in claim 30 wherein the polymeric material is selected from the group consisting of polyamides, polyesters, polyacrylonitrile, polyethylene, polypropylene, polyglycolic acid, polylactic acid, polydioxanone, polyepsilon-caprolactone, and polytrimethylene carbonate.

35. A method of closing a wound in tissue comprising:
a. providing a suture comprising first yarns and second yarns in a braided construction wherein the first yarns include a plurality of filaments comprising a polymeric material, and the second yarns include a plurality of filaments comprising a shape memory material; and
b. passing said suture through the tissue;
c. securing the ends of said suture to approximate the tissue.

36. A method of securing soft tissue to hard tissue comprising:
a. providing a surgical device fabricated from first yarns and second yarns in a braided construction wherein the first yarns include a plurality of filaments comprising a polymeric material and the second yarns include a plurality of filaments comprising a shape memory material;
b. passing said surgical device through the soft tissue;
c. securing said surgical device to the hard tissue;
d. manipulating said surgical device to approximate the soft tissue and hard tissue.

37. The method of claim 36 wherein the step of securing said surgical device to hard tissue further comprises passing said surgical device through an opening formed in the hard tissue.

38. The method of claim 36 wherein the step of securing said surgical device to hard tissue further comprises mounting said surgical device to a suture anchor.

39. The method of claim 36 wherein the step of securing said surgical device to hard tissue further comprises passing the surgical device around hard tissue.

40. The method of claim 36 wherein the step of manipulating said surgical device to approximate the soft tissue and hard tissue comprises forming a knot in said surgical device.

41. A method of approximating hard tissues comprising:
a. providing a multifilament surgical device fabricated from a heterogeneous braid comprising a first yarn and a second yarn in a braided construction wherein the first yarn includes a plurality of filaments comprising a polymeric material; and the second yarn includes a plurality of filaments comprising shape memory material; and
b. manipulating the multifilament surgical device to approximate the hard tissues.

42. A method as in claim 41 further comprising the step of securing the surgical device to hard tissue.

43. A method as in claim 41 wherein said step of providing a multifilament surgical device comprises providing a multifilament surgical device in which said second yarn includes a plurality of filaments comprising nitinol (NiTi).

44. A surgical device comprising:
a suture anchor;
at least one suture secured to the suture anchor, the suture having a braid including of a first set and a second set of continuous and discrete yarns in a braided construction; and
the first set of yarns being heterogeneous yarns containing first and second filaments wherein:
the first set of filaments are made from a polymeric material; and
at least one of the second filaments is made from a shape memory material

45. A surgical device as in claim 44 wherein the second set of yarns are heterogeneous yarns containing first and second filaments wherein:
the first set of filaments are made from a polymeric material; and
at least one of the second filaments is made from a shape memory material.

46. A surgical device as in claim 44 wherein the second set of yarns are homogeneous yarns.

46. A surgical device as in claim 43 wherein the second set of yarns comprise a non-biodegradable material.

47. A surgical device as in claim 43 wherein said polymeric material is selected from the group consisting of polyamides, polyesters, polyacrylonitrile, polyethylene, polypropylene, polyglycolic acid, polylactic acid, polydioxanone, polyepsilon-caprolactone, and polytrimethylene carbonate.

48. A surgical device as in claim 43 wherein the second set of yarns comprise a biodegradable material.

49. A surgical device as in claim 43 wherein the shape memory material is selected from the group consisting of nitinol (TiNi), CuZnAl, CuAlNi and FeNiAl.

50. A surgical device as in claim 43 wherein the shape memory material is nitinol (NiTi).
